(19) United States
(12) Patent Application Publication
(10) Pub. No.: US 2007/0092447 A1
(43) Pub. Date: Apr. 26, 2007

(54) CONTRAST AGENT FOR COMBINED MODALITY IMAGING AND METHODS AND SYSTEMS THEREOF

(75) Inventors: OMAYRA PADILLA DE JESUS, GUILDERLAND, NY (US); STEPHEN JOHNSON LOMNES, PHILADELPHIA, PA (US); EGIDIJUS EDWARD UZGIRIS, SCHENECTADY, NY (US); FLORIBERTUS P.M. HEUKENSFELDT JANSEN, BALLSTON LAKE, NY (US); PAVEL ALEXEYEVICH FOMITCHOV, NEW YORK, NY (US); DEBORAH STUTZ LEE, NISKAYUNA, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/565,786

(22) Filed: Dec. 1, 2006

Related U.S. Application Data
(63) Continuation-in-part of application No. 10/846,062, filed on May 14, 2004.

(51) Int. Cl. A61K 49/00

(52) U.S. Cl. 424/9.1

(57) ABSTRACT

A combined modality imaging system includes a first imaging device of a first modality and a second imaging device of a second modality that is different from the first modality is provided. The first and the second imaging devices are both adapted to interact with a contrast agent. The contrast agent includes a deformable particle that has a geometry that varies in response to an emission from the first imaging device. The deformable particle also includes a fluorescent component and a quenching component separated from the fluorescent component at a characteristic distance.

### CROSS REFERENCE TO RELATED APPLICATIONS

This application claims priority to pending U.S. patent application Ser. No. 10/846,062, entitled “Contrast Agent for Combined Modality Imaging and Methods and Systems Thereof,” filed on May 14, 2004.

### GOVERNMENT INTERESTS

This invention was developed with government support under U.S. Government Contract No. W81XWH-04-1-0602. Accordingly, the U.S. Government has certain rights to this invention.

### BACKGROUND

The invention relates generally to the field of diagnostic imaging and more specifically, to an imaging method and a system that uses contrast agents conjugated with dyes and quenchers for combined modality imaging, (e.g., optical imaging and ultrasound imaging).

In modern healthcare facilities, medical diagnostic and imaging systems are often used for identifying, diagnosing, and treating physical conditions. Diagnostic imaging refers to any visual display of structural or functional patterns of organs or tissues for a diagnostic evaluation. It includes measuring the physiologic and metabolic responses to physical or chemical stimuli. Currently, a number of modalities exist for medical diagnostic and imaging systems including ultrasound systems, optical imaging systems, computed tomography (CT) systems, x-ray systems (including both conventional and digital or digitized imaging systems), positron emission tomography (PET) systems, single photon emission computed tomography (SPECT) systems, and magnetic resonance imaging (MRI) systems. In many instances, final diagnosis and treatment proceed only after an attending physician or radiologist supplement conventional examinations with detailed images of relevant areas and tissues via one or more imaging modalities.

Some imaging systems analyze the molecular processes concomitant with a disease state rather than the anatomy of the subject. This type of imaging is generally referred to as molecular imaging. The subtle changes in physiological activities, which cause change in molecular concentrations of specific substance, may provide early warning signs of diseases. Detecting such changes requires highly sensitive imaging techniques.

At present, molecular imaging may be employed administering a radiopharmaceutical that targets the specific target area to the patient. The decay of the radiopharmaceutical is used to construct an image of the bio-distribution of the agent. While this method is quite sensitive, it suffers from limited spatial resolution and anatomical registration, and has the further drawback of exposing the patient and the doctor to radiation.

In vivo optical imaging provides an alternative form of molecular imaging that operates by passing light of certain wavelengths into a body and subsequently measuring the change in wavelength following contact with the target tissue. For deeper penetration, In vivo optical imaging generally operates in a near infrared part of the wavelength spectrum, or for applications limited to surface (i.e., external tissue or tissue that has been accessed using a surgical technique) or sub-surface targets a wider range of wavelengths may be employed. The advantages of near-surface optical imaging include the high-resolution visual images and the easy interpretability of the images. However, deep tissue in vivo optical imaging has relatively poor spatial resolution and anatomical registration.

Ultrasound imaging is a modality for quickly obtaining images of a patient's anatomy. In operation, an ultrasound imaging system transmits an ultrasound wave into a subject and subsequently receives a reflected wave that is generated at the interface between tissues of different acoustic impedance. The position of the tissue may be calculated based on the time of arrival and approximate velocity of the reflected wave. Thus, ultrasound imaging systems is used to identify the shape and position of certain anatomies. Although US has the advantage of high spatial resolution, the high noise-to-signal ratio requires considerable skill to properly interpret the images.

In view of the advantages and disadvantages of these different imaging modalities, a technique is needed for combining the high molecular sensitivity of functional imaging modalities (e.g., optical imaging) with the spatial resolution of anatomical imaging modalities (e.g., ultrasound).

### BRIEF DESCRIPTION

Provided herein are agents and methods useful in combined modality imaging systems. The agents of the invention are deformable particles, comprising: (i) a shell encasing an internal substance that expands or contracts in response to an ultrasonic stimulus; and (ii) at least one FRET pair comprising a fluorescent component and a quenching component, wherein the fluorescent component and a quenching component are positioned relative to each other so that the FRET pair an enhanced optical signal when the deformable particle transitions from a neutral conformation to a deformed conformation.

In some embodiments the deformable particle includes one or more FRET pairs that emit a perceivable optical signal when the deformable particle is in an expanded conformation and the FRET pair members are positioned at a distance greater than the characteristic distance.

The internal substance may comprise a gas, a fluid, or a combination of gas and fluid that expands in response to an ultrasound transmission. In some embodiments, internal substance comprises air, sulfur hexafluoride, perfluorocarbon (e.g., perfluoropropane, perfluorobutane, perfluoropentane, perfluorohexane, or a perfluorocarbon gaseous precursor), or a polymer. The shell may comprise an amphiphilic substance, for example, a polymer, a protein (e.g., mammalian serum albumin), or a surfactant.

In some embodiments, the surfactant comprises a detergent selected from C12-sorbitan-E20; Polysorbate 20; Polysorbate 80; C16-sorbitan-E20; or C18-sorbitan-E20. The fluorescent component may comprise a fluorophore selected from indocyanine green, cyanine, fluorescein, rhodamine, yellow fluorescent protein, green fluorescent protein, and derivatives thereof.

In some embodiments, both members of the FRET pair are positioned on the outer surface of the shell. In other embodiments, both members of the FRET pair are positioned within the shell. In still other embodiments, one member of the FRET pair is position on the outer surface of the shell and the other member of the FRET pair is positioned within the shell. In some embodiments, the concentration of the quenching component and the concentration of the fluorescent component are substantially equivalent. In other embodiments, the concentration of the quenching component and the concentration of the fluorescent component are substantially equivalent are of unequal fluorescent efficiencies and the relative concentrations are adjusted to off set the unequal fluorescent efficiencies. In some embodiments the shell further comprises a binder (e.g., antibodies, ligands, or nucleic acids) capable of binding to a predetermined target.

Further provided are combined modality imaging systems, comprising an ultrasound imaging device and an optical imaging device; wherein the ultrasound imaging device comprises an ultrasound probe, a data acquisition and processing system, and an operator interface. In some embodiments, the ultrasound imaging device comprises an ultrasound probe including at least one of an ultrasound transducer, a piezoelectric crystal, and a micro-electro mechanical system device.

The combined modality imaging system may include an ultrasound probe comprising an electromagnetic excitation source and an electromagnetic radiation detector. In other embodiments the ultrasound probe comprises a multitude of electromagnetic radiation detectors.

The combined modality imaging system may further include an ultrasound imaging device comprises a display module to provide a visual display of an ultrasound image in at least one of gray-scale mode and color mode, and a printer module to provide a hard copy of an ultrasound image in at least one of gray-scale mode and color mode, a data acquisition module, a data processing module, or an operator interface.

The optical imaging device may include an electromagnetic excitation source adapted to emit electromagnetic radiation into the subject adapted to emit electromagnetic radiation at least between the ranges of about 300 nanometers and about 2 micrometers and an electromagnetic radiation detector (e.g., photo-multiplier tube, a charged-coupled device, an image intensifier, a photodiode, and an avalanche photodiode) adapted to detect electromagnetic radiation emitted from the contrast agent disposed within the subject.

The electromagnetic excitation source may include at least one radiation transmitting device selected from a group consisting of a solid-state light emitting diode, an organic light emitting diode, an arc lamp, a halogen lamp, and an incandescent lamp. In some embodiments, the optical imaging device comprises at least one fiber-optic channel adapted to convey the electromagnetic radiation from the electromagnetic excitation source to the focus area of the subject. The optical imaging device may include at least one fiber-optic channel adapted to convey the electromagnetic radiation emitted by the contrast agent to the electromagnetic radiation detector.

Also provided are methods using combined modality imaging systems, including the steps of: (a) administering a deformable particle to a subject; (b) applying ultrasound waves into the subject toward a region of interest; (c) applying electromagnetic radiation toward the region of interest; detecting ultrasound signals reflected from the region of interest; (d) detecting electromagnetic radiation from deformable particle; and (e) processing the detected ultrasound signals and the detected electromagnetic radiation.

In some embodiments, the processing step includes producing at least one co-registered image. The applying ultrasound waves and detecting ultrasound signals steps may include the steps of engaging an ultrasound probe with the subject, the ultrasound probe comprising at least one of an ultrasound transducer, a piezoelectric crystal, and a micro electro mechanical system device. The disclosed methods may also comprise the steps of emitting electromagnetic radiation from the fluorescent component in response to emissions from an electromagnetic radiation based imaging device; (a) increasing the geometry of the deformable particle in response to a pressure wave by an ultrasound imaging device; and (b) decreasingly absorbing, with the quenching component, a portion of the electromagnetic radiation emitted by the fluorescent component in response to increasing the geometry of the deformable particle.

### FIGURES

These and other features, aspects, and advantages of the present invention may become better understood when the following detailed description is read with reference to the accompanying figures in which like characters represent like parts throughout the figures.

FIG. 1 is a diagrammatical representation of a combined modality imaging system according to aspects of present technique.

FIG. 2 is a diagrammatical representation of an ultrasound imaging system for use in the multiple modality imaging system of FIG. 1.

FIG. 3 is a diagrammatical representation of an optical imaging system for use in the multiple modality imaging system of FIG. 1.

FIG. 4 is a diagrammatical representation of an alternate implementation of a combined modality imaging system, wherein a single unit comprises an ultrasound probe, an electromagnetic excitation source at one side of the ultrasound probe, and an electromagnetic radiation detector at an opposite side of the ultrasound probe.

FIG. 5 is a diagrammatical representation of another alternate implementation of a combined modality imaging system, wherein a single unit comprises the ultrasound probe and electromagnetic radiation detectors located at opposite sides of the ultrasound probe.

FIG. 6 is a diagrammatic representation of an embodiment of a contrast agent for use with a multiple modality imaging system, wherein a multitude of fluorescent component-quenching component pairs are attached to the outer surface of a deformable particle.

FIG. 7 is a diagrammatic representation of an alternate embodiment of the contrast agent for use with a multiple modality imaging system, wherein a multitude of fluorescent component-quenching component pairs are attached to the inner surface of a deformable particle.

FIG. 8 is a diagrammatic representation of another alternate embodiment of the contrast agent for use with a multiple modality imaging system, wherein a multitude of fluorescent and quenching components are disposed within a shell of a deformable particle.

FIG. 9 is a diagrammatic representation of a further embodiment of the contrast agent for use with a multiple modality imaging system, wherein a multitude of fluorescent and quenching components are disposed in individual shells contained one within the other about a central compressible core.

FIG. 10 is a diagrammatic representation illustrating the interaction between ultrasound waves and a single contrast agent particle disposed within a subject.

FIG. 11 is a diagrammatic representation illustrating the interaction between the electromagnetic radiation and a single contrast agent particle disposed within a subject.

FIG. 12 is a diagrammatic representation illustrating the combined interaction between ultrasound waves, electromagnetic radiation, and a single contrast agent particle disposed within a subject.

FIG. 13 is a flowchart illustrating an exemplary method of use of a combined modality imaging system.

FIG. 14 is a flowchart illustrating an exemplary method of operation for a contrast agent according to aspects of the present technique.

FIG. 15 is a shows microscopy images of microbubble made using Optison (Panel A) and Plasbumin-5 (Panel B).

FIG. 16 depicts changes in fluorescence intensity as a function of dye/protein ratio in which the D/P was determined using MALDI-MS.

FIG. 17 shows confocal microscope images of microbubbles in fluorescence mode, in which Panel A shows Cy5.5-ST68 microbubbles; Panel B shows Control HSA microbubbles plus free Cy5.5; and Panel C shows Cy5.5-HSA microbubbles.

FIG. 18 shows the effects of photobleaching. From the first (Panel A) to ninth image frame (Panel B) taken, two self-quenched bubbles have increased fluorescent intensity.

FIG. 19 shows normalized intensities of four bubbles that were measured over nine image frames to observe effects of photobleaching in self-quenched bubbles.

FIG. 20 shows photobleaching of a portion of an individual microbubble. A higher magnification objective lens was used and only a portion of the image was scanned, two halves of two separate bubbles. The results show increased fluorescence upon photobleaching, followed by decreased fluorescence with additional photobleaching.

### DESCRIPTION

To more clearly and concisely describe and point out the subject matter of the claimed invention, the following definitions are provided for specific terms, which are used in the following description and the appended claims. The singular forms “a”, “an”, and “the” include plural referents unless the context clearly dictates otherwise.

As used herein, “amphiphilic substances” generally refer to molecules that have a polar head attached to a hydrophobic tail (e.g., phospholipids, surfactants or certain polymers).

As used herein, the term “binder” refers to a biological molecule that may non-covalently bind to one or more targets in the biological sample. A binder may specifically bind to a target. Suitable binders may include one or more of natural or modified peptides, proteins (e.g., antibodies, antibody fragments, affibodies, or aptamers), nucleic acids (e.g., polynucleotides, DNA, RNA, or aptamers); polysaccharides (e.g., lectins, sugars), lipids, enzymes, enzyme substrates or inhibitors, ligands, receptors, antigens, haptens, and the like. A suitable binder may be selected depending on the sample to be analyzed and the targets available for detection. For example, a target in the sample may include a ligand and the binder may include a receptor or a target may include a receptor and the probe may include a ligand. Similarly, a target may include an antigen and the binder may include an antibody or antibody fragment or vice versa. In some embodiments, a target may include a nucleic acid and the binder may include a complementary nucleic acid. In some embodiments, both the target and the binder may include proteins capable of binding to each other.

As used herein the term “characteristic distance” refers to the distance of separation upon which the donor can transfer its excitation energy to the acceptor through intramolecular coupling (e.g., the “Förster distance”). A typical range for the characteristic distance is between about 2 nanometers to about 6 nanometers.

As used herein, the terms “deformable particle” and “microbubble” generally refer to a small (e.g., about 2 to about 30 micrometers size range), substantially spherical body of fluid, gas, or a combination of fluid and gas encased within a shell. The microbubbles described herein, deform or change geometry in response to ultrasound waves. In some embodiments, the microbubble shell is composed of amphiphilic substances (e.g., a phospholipid, a surfactant, or a polymer). The deformable particles may adopt three states: contracted, neutral, and expanded. The deformable particles adopt the neutral state when external stimulus (e.g., US transmission) is absent. In the neutral state, donor and acceptor components are located relative to each other such that a fluorescent signal from the FRET pair are quenched. When the deformable particle adopts the expanded state in response to external stimuli (e.g., US transmission and radiation emission) the fluorescent signal from the FRET pair is unquenched and may be read by one or more imaging devices.

As used herein, the term “fluorescent component” refers to a fluorophore (e.g., a FRET donor) that transfers its excitation energy to a nearby quenching component (e.g., FRET acceptor chromophore) in a non-radiative manner. Multiple components with appropriate spectral overlaps may comprise the FRET pair. Examples of paired fluorescent components include, without limitation, fluorescein/rhodamine, cyanine3/cyanine5, CFP/YFB, and Alexa488/Alexa555.

As used herein, the term “fluorophore” refers to a chemical compound, which when excited by exposure to a particular wavelength of light, emits light at a longer wavelength. Fluorophores may be described in terms of their emission profile, or “color.” Green fluorophores (for example Cy3, FITC, and Oregon Green) may be characterized by their emission at wavelengths generally in the range of 515-540 nanometers. Red fluorophores (for example Texas Red, Cy5, and tetramethylrhodamine) may be characterized by their emission at wavelengths generally in the range of 590-690 nanometers.

As used herein the term “Förster Resonance Energy Transfer” or “FRET” refers to an energy transfer mechanism occurring between two fluorescent molecules: a fluorescent donor and a fluorescent acceptor (i.e., a FRET pair) positioned within a range of about 1 to about 10 nanometers of each other wherein one member of the FRET pair (the fluorescent donor) is excited at its specific fluorescence excitation wavelength and transfers the fluorescent energy to a second molecule, (fluorescent acceptor) and the donor returns to the electronic ground state.

As used herein, the term “FRET efficiency” refers to the ability of a FRET pair to demonstrate Förster Resonance Energy Transfer. The FRET efficiency is affected by three parameters, specifically (1) the distance between the donor and the acceptor; (2) the spectral overlap of the donor emission spectrum and the acceptor absorption spectrum; and (3) the relative orientation of the donor emission dipole moment and the acceptor absorption dipole moment.

As used herein the term “internal substance” refers to the contents encased in the shell. Representative internal substances include, without limitation, fluids, gases (e.g., sulfur hexafluoride or a perfluorocarbon), or a combination of fluids and gas (e.g., a foam).

As used herein, the term “quenching component” refers to a chromophore that has an adequate spectral overlay with the fluorescent component to be capable of accepting the energy emitted by the fluorescent component when the members of the pair are positioned at the characteristic distance for FRET. This quenching component could either further emit at a longer wavelength in a cascade fashion or quench the energy of the fluorescent component and not emit any further.

As used herein, the term “quenching” refers to partial or full absorption of energy emitted in form of fluorescence by a fluorescent component. The quenching phenomena may occur between two fluorescent components that are the same or substantially the same (e.g., a single cyanine dye) or two fluorescent components that are different (e.g., a cyanine dye and squarine dye).

As used herein the term “shell” refers to the outer surface of the microbubble. The shell may comprise a single or multiple layers (e.g., bilayer) of amphiphilic substances, for example, phospholipids, surfactants, albumin, or polymers. The shell may be, in some embodiments, a micelle in which the polar heads of the amphiphilic substance or substances are positioned on the outer surface and the apolar tails are positioned within the microbubble. In other embodiments, the shell may include a bilayer, in which the polar heads are positioned on the outer surface of the microbubble, two sets of apolar tails are sandwiched between the outer surface polar heads and a second layer of polar heads positioned within the microbubble.

As used herein the term “spectral overlap” generally refers to the range of values where the emission spectrum (i.e., the amount of electromagnetic radiation of each frequency it emits when it is excited) of the donor overlaps the absorption spectrum of the acceptor (i.e., fraction of incident electromagnetic radiation absorbed by the material over a range of frequencies). As used herein the term “surfactant” generally refers to organic compounds that are amphiphilic, which reduce the surface interfacial tension between two liquids. Preferred surfactants assemble into micelles or reverse micelle.

Surfactants may be ionic (i.e., anionic or cationic), non-ionic, and zwitterionic. Examples of anionic surfactants include those compounds based on sulfate, sulfonate or carboxylate anions (e.g., sodium dodecyl sulfate, ammonium lauryl sulfate, or sodium laureth sulfate). Examples of cationic surfactants include cationic compounds based on quaternary ammonium cations (e.g., cetyl trimethylammonium bromide; cetylpyridinium chloride; polyethoxylated tallow amine; benzalkonium chloride; and benzethonium chloride. Examples of Zwitterionic surfactants include dodecyl betaine; dodecyl dimethylamine oxide; cocamidopropyl betaine; Coco ampho glycinate. Examples of nonionic surfactants include alkyl poly(ethylene oxide); and alkyl polyglucosides (e.g., octyl glucoside and decyl maltoside). In some embodiments, the surfactant may comprise members of the sorbitan family, including TWEEN 20 (C12-sorbitan-E20; Polysorbate 20); TWEEN 40 (C16-sorbitan-E20); TWEEN 60 (C18-sorbitan-E20); and TWEEN 80 (C18:1-sorbitan-E20).

As used herein, the term “unquenching” refers to the increase in fluorescence emission due to the decrease or absence of a FRET partner or change in characteristic distance. Thus, unquenching may occur, for example, when there is increase in distance between a donor-acceptor pair resulting in increased fluorescence emission.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

The contrast agents provided herein, which may be referred to as Quenchable Fluorescent Microbubbles (QFMB), may be composed of: a microbubble shell encasing an internal substance, and a fluorescent component. In some embodiments, the fluorescent component is a single fluorescent dye that self-quenches at specific concentrations. In alternative embodiments the fluorescent component is a pair of fluorescent dyes with spectral overlap.

In all embodiments, the fluorescent dye or dyes may be covalently attached to the surface of the microbubble such that the distance between the dyes increase upon expansion of the shell and decreased upon contraction of the shell. Because the energy transfer efficiency is proportional to the inverse of the sixth power of the distance between the dyes, small changes in distance may produce large changes in fluorescence intensity.

In accordance with one aspect of the present invention, provided herein are contrast agents for a combined modality imaging system including a deformable particle that changes geometry (e.g., radius) in response to an emission from the combined modality imaging system. The deformable particle also includes a fluorescent component (e.g., a FRET donor) that is adapted to emit electromagnetic radiation and a quenching component (e.g., a FRET acceptor) separated from the fluorescent component and adapted to absorb a portion of the electromagnetic radiation from the fluorescent component.

Also provided herein are combined modality imaging systems including a first imaging device of a first modality and a second imaging device of a second modality that is different from the first modality. The first and the second imaging devices are both adapted to interact with a contrast agent. The contrast agent includes a deformable particle that has a geometry that varies in response to an emission from the first imaging device. The deformable particle also includes a fluorescent component adapted to emit electromagnetic radiation that is detectable by the second imaging device and a quenching component separated from the fluorescent component at a distance based on the geometry and that is adapted to absorb a portion of the electromagnetic radiation from the fluorescent component.

In accordance with another aspect of the present invention, provided herein are methods of using combined modality imaging systems including administering a contrast agent provided herein to a subject. The deformable particle includes a fluorescent component adapted to emit electromagnetic radiation detectable by an electromagnetic radiation based imaging device and a quenching component that is separated from the fluorescent component at a distance based on a geometry of the deformable particle, wherein the quenching component is adapted to absorb a portion of the electromagnetic radiation emitted by the fluorescent component. The quenching component may also produce an energy transfer without emission of electromagnetic radiation from the fluorescent component by a fluorescent resonance energy transfer mechanism.

The method of use of the combined modality imaging system also includes applying ultrasound waves from an ultrasound imaging system on to a region of interest of an ultrasound probe in a region of interest on the subject, applying electromagnetic radiation using an electromagnetic excitation source on the region of interest, detecting the reflected ultrasound signals using the ultrasound probe, detecting the electromagnetic radiation from the contrast agent using an electromagnetic radiation detector, processing the detected ultrasound signals and the electromagnetic radiation to obtain at least one image, and optionally displaying the images from the combined modality imaging system.

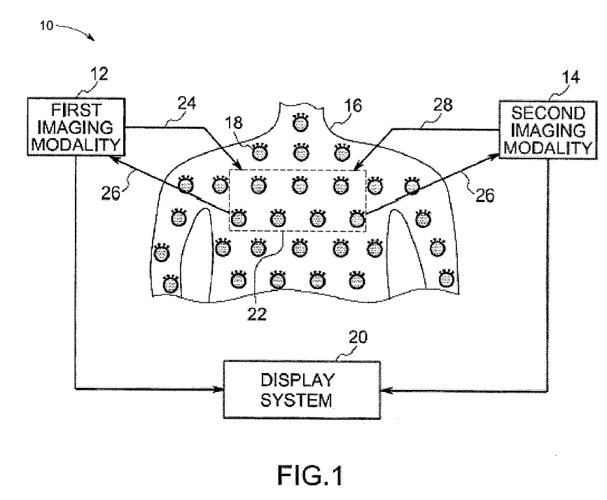

Turning now to the drawings, and referring to FIG. 1, a combined modality imaging system 10 is illustrated schematically as including a first imaging modality 12, a second imaging modality 14, a subject 16 to which a contrast agent 18 has been administered, and a display system 20 capable of displaying the image from the first and second imaging modalities.

The contrast agents provided herein may be administered to a subject “parenterally”, for example, by intravenous, intramuscular, intraperitoneal, intrasternal, subcutaneous and intraarticular injection or infusion. The contrast agent may, following administration, localize at regions of interest, such as tumor tissue, to enhance imaging of those regions of interest.

As discussed in detail below, certain embodiments of the contrast agent 18 comprise a deformable particle having a fluorescent component and a quenching component offset from the fluorescent component, such that variation in the geometry (e.g., expansion or contraction) of the deformable particle changes the distance between the fluorescent and quenching components. In operation, the electromagnetic radiation emitted from the contrast agent 18 varies with distance between the fluorescent and quenching components. In some embodiments, greater distance results in relatively more emitted electromagnetic radiation and a smaller distance results in relatively less radiation.

According to aspects of the present technique, the first imaging modality 12 focuses pressure waves 24 at a desired frequency (e.g., the range of about 0.1 MHz to about 50 MHz) onto a region of interest 22 on the subject 16 and retrieves reflected pressure waves 26 from the region of interest 22 to obtain an image. For example, one embodiment of the first imaging modality 12 includes an ultrasonic probe 32 that transmits and receives ultrasound waves in a region of interest 22. In the region of interest 22, the pressure waves 24 functions to alter the geometry (e.g. cause expansion) of the contrast agent 18, thereby modulating the fluorescence emitted by the contrast agent 18 at the frequency of the pressure waves 24. Embodiments of the second imaging modality 14 detect this fluorescent modulation to generate an optical molecular image that is substantially localized based on the region of interest 22.

In operation, the second imaging modality 14 transmits electromagnetic radiation 28 onto the region of interest 22 and then utilizes the interaction between the first imaging modality 12, the contrast agent 18, and the electromagnetic radiation 28 to generate an image. The display system 20 may display the images from the two different modalities either separately or as a composite image where the images are superimposed one on top of the other.

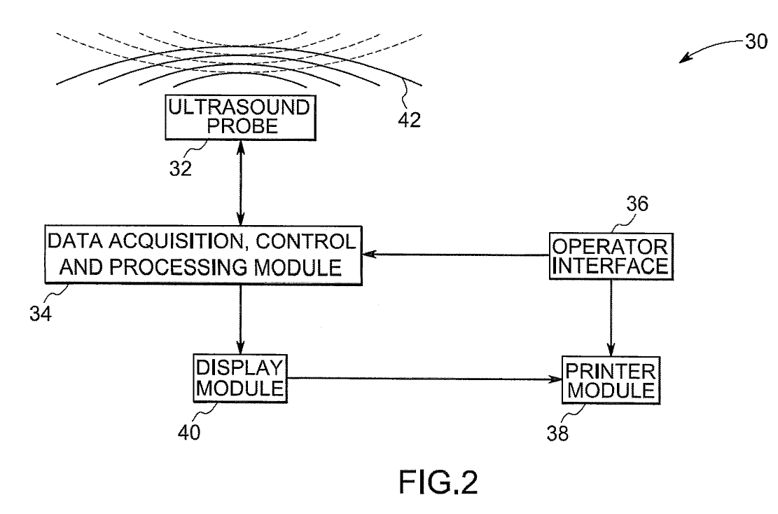

The present technique combines the advantages of a high molecular sensitivity of functional imaging modalities (e.g., optical imaging) with the advantages of a high spatial resolution of anatomical imaging modalities (e.g., ultrasound imaging) to improve image quality and diagnosis. FIG. 2 illustrates an exemplary first imaging modality 12 as an ultrasound system 30 that includes an ultrasound probe 32, a data acquisition and processing module 34, an operator interface 36, a printer module 38, and a display module 40.

In operation, the ultrasound probe 32 sends and receives ultrasound waves 42 from a region of interest on the subject 16. The ultrasound probe 32, according to aspects of present technique, includes at least one of an ultrasound transducer, a piezoelectric crystal, an opto-acoustic transducer and a micro-electro mechanical system device, for example, a capacitive micro-machined ultrasound transducer (cMUT). The relatively high frequency of ultrasound also facilitates relatively focused targeting of the ultrasound waves 42. During the operation of the ultrasound system 30, the ultrasound waves 42 reflected from the subject carry information about the thickness, size, and location of various tissues, organs, tumors, and anatomical structures in relation to the transmitted ultrasound wave. In certain embodiments, the ultrasound probe 32 may be hand-held or mechanically positioned using a robotic assembly.

The data acquisition, control, and processing module 34 sends and receives information from the ultrasound probe 32. It controls the strength, the width, the duration, and the frequency of the ultrasound waves 42 transmitted by the ultrasound probe 32 and decodes the information contained in the ultrasound waves 42 reflected from the region of interest 22 to discernable electrical and electronic signals. Once the information is obtained, the image of the object located within the region of interest 22 of the ultrasound probe 32 is reconstructed.

The operator interface 36 may include a keyboard, a mouse, and other user interaction devices. The operator interface 36 may be used to customize the settings for the ultrasound examination, and for effecting system level configuration changes. The operator interface 36 is connected to the data acquisition, control, and processing module 34 and to the printer module 38. The printer module 38 is used to produce a hard copy of the obtained ultrasound image in either gray-scale or color. The display module 40 presents the reconstructed image of an object within the region of interest 22 on the subject 16 based on data from the data acquisition and processing module 34.

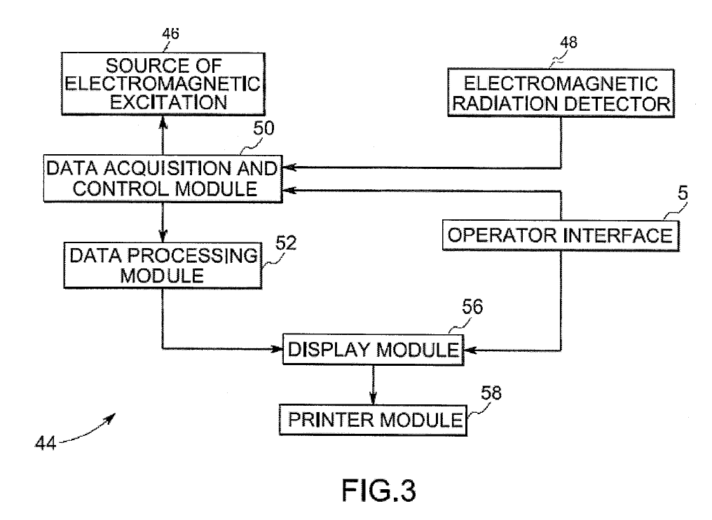

FIG. 3 illustrates an exemplary optical imaging system 44. In certain embodiments, the optical imaging system 44 operates in conjunction with the ultrasound imaging system 30 of FIG. 2. The illustrated optical imaging system 44 includes an electromagnetic excitation source 46, an electromagnetic radiation detector 48, a data acquisition and control module 50, a data processing module 52, an operator interface 54, a display module 56, and a printer module 58. As discussed in further detail below, the optical imaging system 44 records the interaction between the ultrasound system 30, a solution of the contrast agent 18 injected within and located in the region of interest 22 of the subject 16, and the electromagnetic radiation from the electromagnetic excitation source 46.

The illustrated electromagnetic excitation source 46 has at least one of a solid state light emitting diode (LED), an organic light emitting diode (OLED), a laser, an incandescent lamp, a halogen lamp, an arc lamp and any other suitable light source. For example, the electromagnetic excitation source 46 may emit radiation between the ranges of about 300 nanometers and about 2 micrometers that is matched to the absorption wavelength of a fluorescent component. Certain embodiments of the electromagnetic excitation source 46 emit electromagnetic radiation whose intensity may be time invariant, a sinusoidal variation, a pulse variation, or time varying. The electromagnetic radiation may also comprise a single wavelength or many wavelengths covering a spectrum from about 300 nanometers to about 2 micrometers. Fiber-optic channels, such as an optic fiber and bundles of optic fibers may also be used to provide illumination from the electromagnetic excitation source 46 to the region of interest 22.

The illustrated electromagnetic radiation detector 48 has at least one of a photomultiplier tube, a charged-coupled device, an image intensifier, a photodiode, an avalanche photodiode, and any suitable device that may convert a time-varying flux of electromagnetic radiation to a time-varying electrical signal. An array of optical fibers may also be extended from the electromagnetic radiation detector 46 to the vicinity of the region of interest 22 to collect electromagnetic radiation. For example, the optical fibers may be mounted either directly on the subject 16 or near the surface of the subject 16.

The illustrated data acquisition and control module 50 sends control signals to the electromagnetic excitation source 46 and receives the optical signals from the electromagnetic radiation detectors 48. The data acquisition and control module 50 also communicates with the data processing module 52 and the user interface module 54. The data processing module 52 re-constructs an image using the information obtained from the electromagnetic radiation detector 48. The user interface module 54 is used to make changes to the configuration of the optical imaging system 44 and to provide control commands to the display module 56 and the printer module 58.

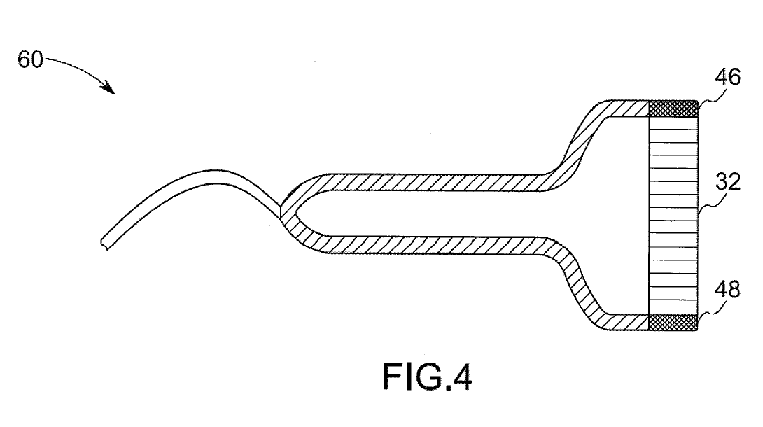

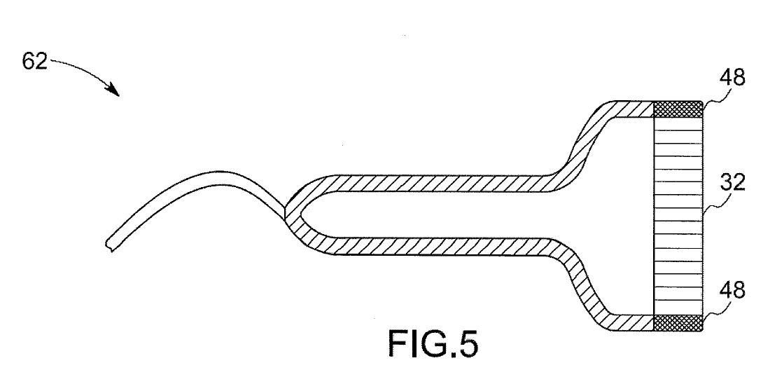

In certain embodiments, the combined modality imaging system 10 includes the functionalities of both the ultrasound and the optical imaging systems as described in detail above. FIGS. 4 and 5 are exemplary embodiments of such combined modality imaging systems. The embodiment of FIG. 4 comprises a single unit having the ultrasound probe 32 of the ultrasound imaging system 30 located at the center of the single unit, and the electromagnetic excitation source 46 and the electromagnetic detector 48 of the optical imaging system 44 located at opposite sides of the single unit. The embodiment of FIG. 5 comprises a single unit having the ultrasound probe 32 of the ultrasound imaging system 30 at the center of the single unit, and a pair of the electromagnetic radiation detectors 48 of the optical imaging system 44 at opposite sides of the ultrasound probe 32.

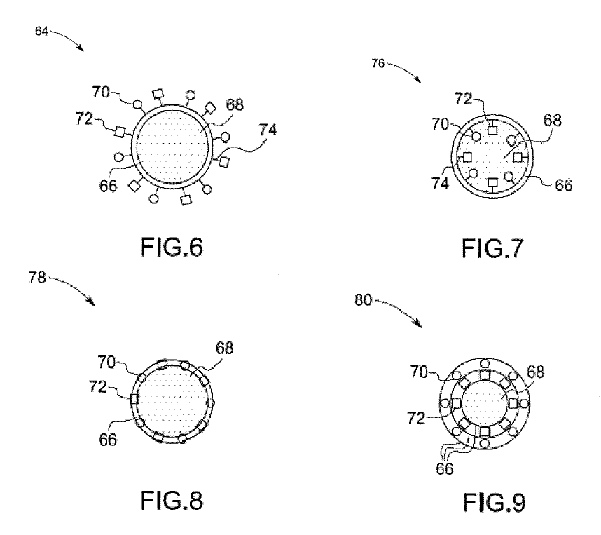

As described below with reference to FIGS. 6-9, the foregoing imaging systems 10, 30, and 44 interact with a variety of different embodiments of contrast agents. In general, the contrast agents provided herein, include a deformable shell and a fluorescent-quencher pair.

FIG. 6 is a diagrammatic illustration of an embodiment 64 of the contrast agent 18 that comprises a deformable particle, including a shell 66 and an internal substance 68 disposed within the shell 66. The deformable particle also includes one or a multitude of fluorescent component 70 and quenching component 72 pairs, each component attached to the outer surface of the deformable particle.

FIG. 7 is a diagrammatic illustration of an alternate embodiment 76 of the contrast agent 18 comprising a deformable particle including a shell 66 and an internal substance 68 disposed within the shell 66. The deformable particle also includes at least one of a fluorescent component 70—quenching component 72 pair disposed within the shell 66 of the deformable particle, each component attached to the inner surface of the deformable particle by means of a linker component 74.

FIG. 8 is a diagrammatic illustration of another alternate embodiment 78 of the contrast agent 18 that comprises a deformable particle, wherein a multitude of the fluorescent component 70 and quenching component 72 pairs form the shell 66 of the deformable particle.

FIG. 9 is a diagrammatic illustration of a further embodiment 80 of the contrast agent 18, wherein at least of the fluorescent component 70 and the quenching component 72 is disposed separately in multiple layers of the deformable particle and the inner shell comprises a compressible core. In each of the foregoing embodiments, the sound wave 42 (e.g., an ultrasound wave) changes the geometry of the deformable particle, thereby changing the distance between the fluorescent component—quenching component pairs. The figures discussed below further describe the composition and the interaction of the contrast agents 18 with the ultrasound imaging system 30 illustrated in FIG. 2 and the optical imaging system 44 illustrated in FIG. 3.

The shell 66 of the deformable particle includes at least one of a polymer, a protein, and an amphiphilic molecule (e.g., phospholipids, proteins, or surfactants) containing both hydrophobic and hydrophilic regions. The amphiphilic molecule may include at least one surfactant of an ionic nature or a non-ionic nature, wherein the surfactant includes at least one functional group that provides at least one reactive handle (e.g., a hydroxyl group that was reactive to provide an amine capable of reacting with NHS activated ester of the dye or an amine of the lysine groups in proteins capable of reacting with NHS activated ester dyes) for a continued chemical modification. Thus, the reactive handle may be used to attach a chemical moiety, including but not limited to binders or dyes, to the deformable particle.

The internal substance 68 disposed within the shell 66 is compressible, and in certain embodiments, may include at least one of air, sulfur hexafluoride, a perfluorocarbon, foam, a gas precursor, and polymer.

The fluorescent component 70 comprises a fluorescent dye. For example, the fluorescent dye may include indocyanine green (ICG), cyanine 5.5 (CY5.5), cyanine 7.5 (CY7.5), fluorescein, rhodamine, yellow fluorescent protein (YFP), green fluorescent protein (GFP), fluorescein isothiocyanate (FITC), and their derivatives.

The fluorescent component 70 absorbs electromagnetic radiation at an incident wavelength and emits electromagnetic radiation at a longer wavelength. The quenching component 72 absorbs the electromagnetic radiation at the wavelength emitted by the fluorescent component 70. One function of the fluorescent component 70 is to maximize the light output from the region of interest 22 of the ultrasound probe 32. One function of the quenching component 72 is to maximize the signal-to-noise ratio by minimizing the intensity of fluorescent light produced by particles that are not near the region of interest on the subject 16.

If the distance between the fluorescent component 70 and the quenching component 72 is less than a characteristic distance and the electromagnetic radiation from the electromagnetic excitation source 46 is incident on the region of interest on the subject 16, then the electromagnetic radiation emitted by the fluorescent component 70 (after absorbing the incident electromagnetic radiation from the electromagnetic excitation source 46 illustrated in FIG. 3) is quenched by the quenching component 72.

When mechanism of action is FRET quenching occurs when the quenching component 72 absorbs most of the electromagnetic radiation emitted by the fluorescent component 70. Quenching may also occur by a FRET mechanism where the quenching component 72 absorbs the energy from the fluorescent component 70 without any emission of electromagnetic radiation from the fluorescent component 70. As a result, there is a weak output of light from the contrast agent 18 that is insufficient to be detected by the electromagnetic radiation detector 48. At this point, the contrast agent 18 is said to be in an OFF state. A typical dimension of the contrast agent in its OFF state is less than 15 micrometers in diameter.

If the distance of separation between the fluorescent component 70 and the quenching component 72 at least exceeds the characteristic distance, called the Förster distance, and the electromagnetic radiation from the electromagnetic excitation source 46 is incident on the region of interest 22 of the subject 16, then the electromagnetic radiation emitted by the fluorescent component 70 would not be absorbed by the quenching component 72 and there is light output from the contrast agent 18. At this state, the contrast agent 18 is said to be in an ON state.

The increase in the distance of separation between the fluorescent component 70 and the quenching component 72 is effected when the contrast agent 18 is subjected to ultrasound waves 42 from the proposed ultrasound imaging system 30 illustrated in FIG. 2. Under the influence of acoustic pressure, such as ultrasound waves 42 from an ultrasound imaging system 30, the contrast agent 18 undergoes a change in geometry. In certain embodiments, the ultrasound waves 42 increase the volume of the contrast agent 18. Due to the pulsed nature of the ultrasound waves 42, the contrast agent 18 undergoes repeated compression and expansion resulting in a volume change, which may be of the order of 300% in certain embodiments. The change in volume causes a change in the distance of separation between the fluorescent component 70 and the quenching component 72. Accordingly, there is modulation of the light output every time an ultrasound wave 42 interacts with the contrast agent 18. Therefore, this light output enables the data acquisition and control module 50 of the proposed optical imaging system 44 to collect optical data through the electromagnetic radiation detectors 48 and to process the optical data with the data processing module 52. The data processing module 52 of the optical imaging system 44 computes this optical data to obtain an optical image that is co-registered with the ultrasound image from the ultrasound system 30 illustrated in FIG. 2.

The quenching component 72 comprises at least one of known quenching entities and derivatives thereof. The aforementioned fluorescent component may be self-quenching at a suitable molecular concentration and separation level characteristic for that fluorescent component.

The contrast agent 18 may also include a binder conjugated to the deformable particle, where the binder has a preferential affinity for a biochemical marker (e.g., antibody, antibody fragement, receptor, ligand, or small molecule). In some embodiments, the binder may be covalently attached to the deformable particle through a reactive handle present on the shell. In those embodiments wherein the contrast agent 18 includes a binder, the contrast agent may preferentially target abnormal tissue due to the differences in the expression patterns of the biomarker between the abnormal tissue and a normal tissue.

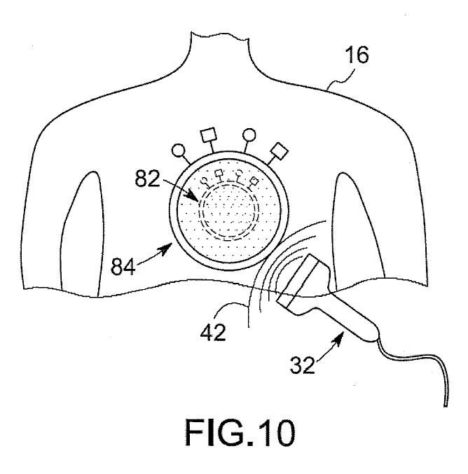

FIG. 10 is an exemplary illustration of interaction between ultrasound waves 42 from the ultrasound imaging system 30 and a contrast agent 18. Before the ultrasound wave 42 hits the contrast agent 18, the contrast agent 18 is in its ground or unexcited state 82, wherein the distance of separation between the fluorescent component 70 and the quenching component 72 is less than the characteristic distance. When the ultrasound waves 42 hits the contrast agent 18, the contrast agent 18 expands, increasing the distance of separation between the fluorescent component 70 and the quenching component 72. At this stage, the contrast agent 18 is in an excited stage 84, wherein the distance of separation between the fluorescent component 70 and the quenching component 72 at least exceeds the characteristic distance. Consequently, the quenching component does not quench the fluorescent component such that the deformable particle generates an increased optical signal relative to a similar contrast agent in the neutral position.

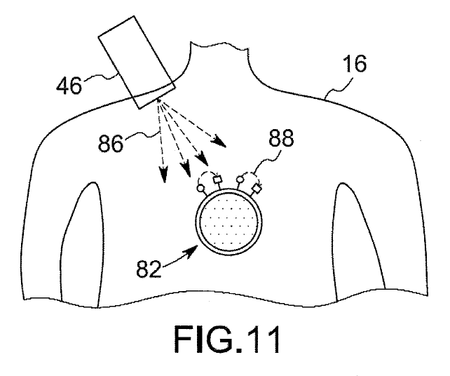

FIG. 11 is an exemplary illustration of interaction between a single contrast agent 18 and an electromagnetic excitation source 46 from the optical imaging system 44. The electromagnetic excitation source 46 emits electromagnetic radiation 86 between ranges of about 300 nanometers and about 2 micrometers and matched to the absorption wavelength of the contrast agent 18. The fluorescent component 70 absorbs the incident electromagnetic radiation 86, and emits electromagnetic radiation 88 at a longer wavelength. However, since the distance between the fluorescent component 70 and the quenching component 72 is less than the characteristic distance there is maximum energy transfer between the two components. Because there is maximum energy transfer, the quenching component 72 absorbs the electromagnetic radiation 88 emitted by the fluorescent component 70 and there is a weak output in the form of an electromagnetic radiation from the contrast agent 18 and the quenching component quenches the fluorescent component such that the deformable particle generates a decreased optical signal relative to a similar contrast agent in the neutral position.

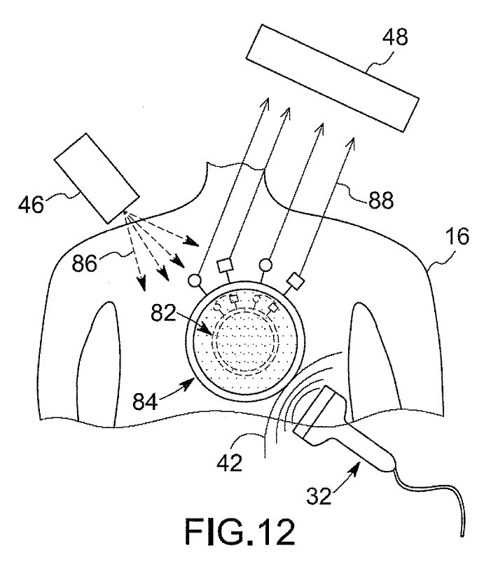

FIG. 12 illustrates the combined interaction of the ultrasound and optical imaging modalities described hereinabove with a contrast agent 18. In operation, the electromagnetic radiation 86 from the electromagnetic excitation source 46 is incident on a contrast agent 18 in the region of interest 22 of an ultrasound probe 32. First, the ultrasound waves 42 from an ultrasound probe 32 strike the contrast agent 18, thereby causing a change in the state of the contrast agent 18 from an OFF state 82 to an ON state 84, resulting in an expansion of the deformable particle of the contrast agent 18. As discussed above, the expansion causes an increase in the distance of separation between the fluorescent component 70 and the quenching component 72. Because the electromagnetic radiation 86 is incident on the fluorescent component 70 of the excited contrast agent 84, the electromagnetic radiation detector 48 of the optical imaging system 44 detects the output in the form of an electromagnetic radiation 88 emitted by the contrast agent 18.

In an alternative embodiment of the present technique, the contrast agent 18 may behave differently when subjected to an ultrasound pulse as discussed below. Consider when the contrast agent is subjected to an ultrasound pulse. Specifically, in this alternative embodiment, the contrast agent 18 may change geometry in a manner in which the volume of the contrast agent increases with each ultrasound wave that passes through the contrast agent 18. When the ultrasound wave 42 is turned off, the volume of the contrast agent 18 does not shrink back to its original state abruptly. Instead, the volume of the contrast agent 18 undergoes a gradual reduction in its geometry until its ground state is reached.

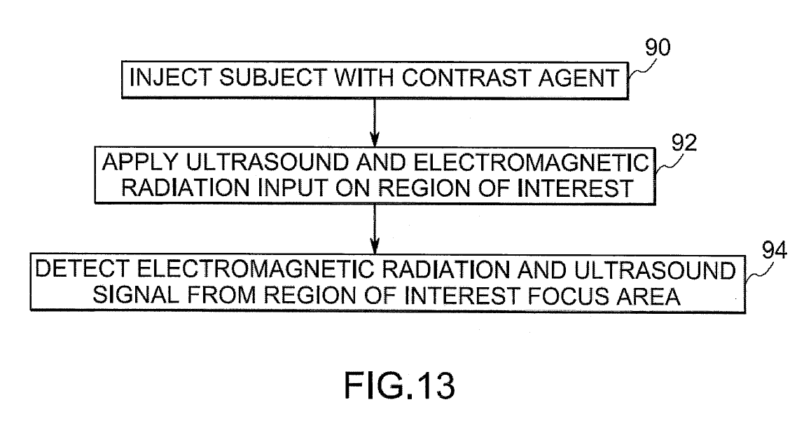

FIG. 13 illustrates an exemplary method of use of the combined modality imaging system 10 illustrated in FIG. 1. The method involves administering (e.g., by injection) a contrast agent 18 to a subject at step 90. After a sufficient amount of time, the contrast agent 18 flows through the subject 16 to the region of interest 22, where the imaging is to be performed to aid in a diagnosis. At step 92, the inputs (ultrasound waves and electromagnetic radiation) from the combined modality imaging system 10 are applied onto the region of interest 22 of the subject 16. The contrast agent 18 interacts with both the ultrasound imaging system 30 and the optical imaging system 44 in the manner described in the sections herein above. At step 94, the combined modality imaging system 10 detects the electromagnetic radiation emitted by a multitude of the fluorescent component 70 of the contrast agent 18 as well as the ultrasound waves 42 reflected from the focus area of the subject.

In one embodiment, a simultaneous mapping of the radiographic ultrasound image obtained from the ultrasound imaging system 12 with the concentration of the contrast agent, which is measured by an intensity of electromagnetic radiation emitted by the contrast agent 18 and detected by electromagnetic radiation detectors 48 in the optical imaging system 14. This intensity of electromagnetic radiation may be the modulated intensity as received or it may be a modified intensity based on an estimate of the attenuation caused by any intermediate tissue or organ. The display may be separate displays or a composite display wherein the images from the two different modalities are superimposed one over the other. Finally, at optional step 96, the co-registered images from the first imaging modality 12 and the second imaging modality 14 are displayed.

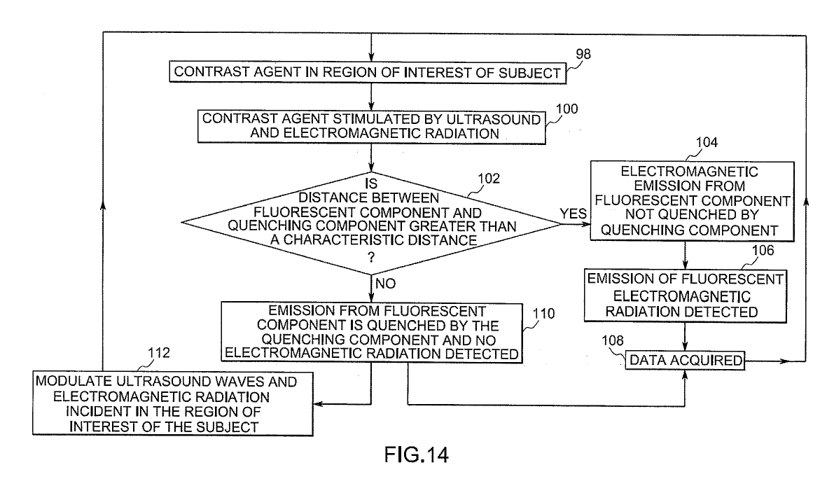

FIG. 14 illustrates a method of operation for a contrast agent (e.g., as illustrated in FIGS. 6-9) and combined modality imaging system. At step 98, the contrast agent 18 initially accumulates in a region of interest 22 on a subject 16. At step 100, the contrast agent 18 excites or becomes stimulated in response to ultrasound and electromagnetic radiation. For example, an input in the form of an electromagnetic radiation 28 from the combined modality imaging system 10 may be applied on the region of interest 22 containing the contrast agent 18, such that there is emission of electromagnetic radiation from the fluorescent component 70. The quenching component absorbs a portion of the electromagnetic radiation emitted by the fluorescent component 70. As discussed in detail above, the amount of absorption depends on the distance of separation between the fluorescent component and the quenching component. The distance of separation is governed by the geometry of the deformable particle.

Furthermore, at step 100, when an input in the form of an ultrasound wave is directed towards the region of interest 22, the deformable particle undergoes a change in geometry that results in a change in the distance of separation between the fluorescent component and the quenching component. Step 102, represents the dependence on the distance of separation as a factor that determines whether the contrast agent 18 emits electromagnetic radiation or not. The flow proceeds to step 104 if the distance of separation at least equals a characteristic distance, called the Förster distance. The fluorescent component 70 emits electromagnetic radiation that is not absorbed by the quenching component 72. As shown in step 106, the contrast agent 18 emits electromagnetic radiation detectable by an electromagnetic radiation detector. If the distance of separation is less than the Förster distance, then the flow proceeds from step 100 to step 110. During this phase, the emitted electromagnetic radiation from the fluorescent component is quenched by the quenching component by any one of the quenching mechanisms described in detail above.

At step 112, the ultrasound wave 32 from the combined modality imaging system may be suitably modified to increase the distance of separation. Furthermore, at step 112, the wavelength of the electromagnetic radiation from the electromagnetic excitation source 46 may be modified to facilitate maximum absorption by the fluorescent component. Step 108 represents the continuous acquiring of data irrespective of whether there is emission of electromagnetic radiation from the contrast agent. The process is repeated until sufficient data has been acquired.

In accordance with certain embodiments of the present technique, a method of manufacture of a contrast agent (e.g., as illustrated in FIGS. 6-9) may comprise the steps discussed in detail below. The contrast agent 18 includes a deformable particle that has a shell 66 and an internal substance 68 along with at least one of a fluorescent component 70 and a quenching component 72. The method involves using a template as a temporary core that facilitates the manufacture of contrast agents of uniform dimension. In certain embodiments, the shell 66 may be assembled on top of the template by the formation of covalent bonds, such as covalent bonds made by a cross-linking by partial denaturation of a protein, a cross-linking with a polyfunctional linker, cross-linking with a polymerizable group, and any combinations thereof.

Alternatively, in other embodiments, the shell 66 may be stabilized by at least one non-covalent interaction, such as a hydrophobic interaction, a hydrophilic interaction, or an ionic interaction. The covalent bond has at least one of a biodegradable linkage and a non-biodegradable linkage. The deformable particle is thus formed. Individual components containing functional handles that allow for further modification of the deformable particle are introduced. These functional handles facilitate the attachment of the fluorescent component 70 and the quenching component 72 to the shell 66. Alternately, in another embodiment, the fluorescent component 70 and the quenching component 72 may attach directly to the shell 66. One of the fluorescent component 70 and the quenching component 72 are introduced to the deformable particle for the formation of the contrast agent 18.

### EXAMPLES

### Example 1

### Preparation of Non-fluorescent Microbubbles

Although non-fluorescent microbubbles are commercially available, we synthesized non-fluorescent microbubbles as follows. Two different scaffolds were selected for the preparation of non-fluorescent microbubbles: a surfactant-based system composed of Tween 80 and Span 60 (ST68), and the Human Serum Albumin (HSA) protein. The non-ionic surfactant based system, which has been previously studied, is stabilized by hydrophilic/hydrophobic interactions of the surfactant forming stable micellar-like type of systems. This system would provide flexibility to manipulate the density of dyes on the shell by the addition of different ratios of fluorescently labeled- versus non-labeled surfactants, assuming that these would arrange evenly around the shell due to the micellar type of system.

The commercially available microbubble Optison® is based on HSA, which includes multiple lysine (Lys) groups that may be used for covalent attachment of the fluorophore to the scaffold. This system is believed to be formed due to denaturation of the protein under sonication conditions and to be stabilized by disulfide bonds formed. However, in the case of the HSA scaffold, even though the number of dyes attached to the protein may be changed, the primary structure of the lysines positioned along the protein chain, the random labeling and the conformation acquired after denaturation of the protein may determine the orientation of the dyes.

### Example 1A

### Preparation of Surfactant-Based Microbubbles

The surfactant solution was prepared as follows: 1.48 g of Span 60 and 1.5 g of NaCl were ground together in a mortar with a pestle until homogeneous mixture was formed. Then, 10 mL of phosphate buffer solution (PBS) solution were added and mixture was mixed to slurry. The slurry was poured into a 50 mL beaker. 10 mL of PBS were added to the mortar followed by the addition of 1 mL of Tween 80. These two were mixed and then combined with the slurry. The mortar was rinsed with an additional 30 mL of PBS and added to the beaker.

For the preparation of ST68 microbubbles the parameters that were changed include, volume of the solution, sonication time, continuous vs. non-continuous sonication, sonication intensity, type of bath in which solution was immersed during sonication and depth of horn tip into solution. The sonicator used (Sonics and Materials, Inc. VCX 750 Model, CT, USA) was set to a frequency 20 KHz. The ultrasound contrast agent Optison® (GE Healthcare) was used as a benchmark to compare the bubbles prepared.

The conditions tested are summarized in Table 1. As the first 17 samples were tested, only samples 14 and 17 showed promising results. These results suggested that higher intensity sonication improved microbubble formation. However, when changing the position of the tip of the horn from the center of the solution to the surface of the solution, better results were obtained even when sonicating for shorter time. Samples 18 to 29 were tested using same conditions as for samples 14-17, except for the positions of the tip of the horn. Samples 20, 23 and 26 showed the thickest layers of microbubbles. These results may be due to better incorporation of air into the solution to by positioning the tip of the horn close to the surface. Two important elements for the formation of microbubbles were (1) higher intensity sonication and (2) sonication at the surface of the solution. A summary of conditions for preparation of ST68 microbubbles is provided below, in which a=pulse of 2 sec sonication, 0.5 sec pause.

TABLE 1

| | | | | | Son. | | Probe | | |
|---|---|---|---|---|---|---|---|---|---|
| | | Volume | Son. | Cont/Not | intensity | Horn tip | size | | |
| Sample | Solvent | (mL) | time | cont | (%) | position | (in.) | Bath | Result |
| 1 | PBS | 10 | 3 min | C | 21 | center | ⅛ | NB | No MBs |
| 2 | PBS | 10 | 10 min | C | 21 | center | ⅛ | NB | No MBs |
| 3 | PBS | 8 | 15 min | NC | 38 | center | ⅛ | NB | MBs |
| | | | (2 sec, | | | | | | low |
| | | | 0.5 sec)^{a} | | | | | | yield |
| 4 | PBS | 8 | 15 min | C | 38 | center | ⅛ | ice | No MBs |
| | | | | | | | | water | |
| 5 | PBS | 8 | 15 min | C | 38 | center | ⅛ | NB | No MBs |
| 6 | PBS | 4 | 3 min | C | 21 | center | ⅛ | NB | No MBs |
| 7 | PBS | 4 | 3 min | C | 30 | center | ⅛ | NB | No MBs |
| 8 | PBS | 4 | 3 min | C | 38 | center | ⅛ | NB | No MBs |
| 9 | PBS | 4 | 5 min | C | 21 | center | ⅛ | NB | No MBs |
| 10 | PBS | 4 | 5 min | C | 30 | center | ⅛ | NB | No MBs |
| 11 | PBS | 4 | 5 min | C | 38 | center | ⅛ | NB | No MBs |
| 12 | PBS | 4 | 10 min | C | 21 | center | ⅛ | NB | No MBs |
| 13 | PBS | 4 | 10 min | C | 30 | center | ⅛ | NB | No MBs |
| 14 | PBS | 4 | 10 min | C | 38 | center | ⅛ | NB | MBs |
| 15 | PBS | 4 | 15 min | C | 21 | center | ⅛ | NB | No MBs |
| 16 | PBS | 4 | 15 min | C | 30 | center | ⅛ | NB | No MBs |
| 17 | PBS | 4 | 15 min | C | 38 | center | ⅛ | NB | MBs |
| 18 | PBS | 4 | 3 min | C | 21 | surface | ⅛ | NB | No MBs |
| 19 | PBS | 4 | 3 min | C | 30 | surface | ⅛ | NB | No MBs |
| 20 | PBS | 4 | 3 min | C | 38 | surface | ⅛ | NB | MBs |
| 21 | PBS | 4 | 5 min | C | 21 | surface | ⅛ | NB | No MBs |
| 22 | PBS | 4 | 5 min | C | 30 | surface | ⅛ | NB | MBs |
| 23 | PBS | 4 | 5 min | C | 38 | surface | ⅛ | NB | MBs |
| 24 | PBS | 4 | 10 min | C | 21 | surface | ⅛ | NB | No MBs |
| 25 | PBS | 4 | 10 min | C | 30 | surface | ⅛ | NB | MBs |
| 26 | PBS | 4 | 10 min | C | 38 | surface | ⅛ | NB | MBs |
| 27 | PBS | 4 | 15 min | C | 21 | surface | ⅛ | NB | No MBs |
| 28 | PBS | 4 | 15 min | C | 30 | surface | ⅛ | NB | MBs |
| 29 | PBS | 4 | 15 min | C | 38 | surface | ⅛ | NB | MBs |

### Example 1B

### Preparation of Human Serum Albumin-Based Microbubbles

The initial conditions explored for microbubbles formation were done using HSA solutions that were prepared using lyophilized HSA from Sigma (Cat #: A9511-25G). The parameters that were changed include the solvent used to dissolve the HSA (85 mM NaCl, PBS solution), volume of the solution, sonication time, continuous vs. non-continuous sonication, sonication intensity, size of the probe, type of bath in which solution was immersed during sonication and depth of horn tip in solution.

The initial conditions tried yielded two results. Either unstable large bubbles were formed, which would continuously grow in size after sonication until bursting back into the HSA solution, or the protein would denature and form a gel. The results are summarized below in Table 2 for summary of results.

TABLE 2

| | | Son. | Cont | Son. | | Probe | | |
|---|---|---|---|---|---|---|---|---|
| | | time | Not | intensity | Horn tip | size | | |
| Solvent | Volume (mL) | (min) | cont | (%) | position | (in.) | Bath | Result |
| 85 mM | 10 | 3 | C | 40 | center | ⅛ | NB | gelled |
| NaCl pH | | | | | | | | |
| 7.2 | | | | | | | | |
| PBS pH | 10 | 3 | C | 40 | center | ⅛ | NB | gelled |
| 7.4 | | | | | | | | |
| 85 mM | 10 | 1 | C | 100 | center | ½ | NB | No |
| NaCl pH | | | | | | | | MBs |
| 7.2 | | | | | | | | |
| 85 mM | 10 | 1.5 | C | 100 | center | ½ | NB | No |
| NaCl pH | | | | | | | | MBs |
| 7.2 | | | | | | | | |
| 85 mM | 10 | 2.5 | C | 100 | center | ½ | NB | No |
| NaCl pH | | | | | | | | MBs |
| 7.2 | | | | | | | | |
| 85 mM | 10 | 3 | C | 100 | center | ½ | ice | No |
| NaCl pH | | | | | | | water | MBs |
| 7.2 | | | | | | | | |
| 85 mM | 1.5 | 2 | C | 39 | center | ⅛ | NB | No |
| NaCl pH | | | | | | | | MBs |
| 7.2 | | | | | | | | |
| 85 mM | 1.5 | 3 | C | 39 | center | ⅛ | NB | gelled |
| NaCl pH | | | | | | | | |
| 7.2 | | | | | | | | |
| 85 mM | 1.5 | 2 | C | 39 | center | ⅛ | tap | No |
| NaCl pH | | | | | | | water | MBs |
| 7.2 | | | | | | | | |
| 85 mM | 1.5 | 3 | C | 39 | center | ⅛ | tap | No |
| NaCl pH | | | | | | | water | MBs |
| 7.2 | | | | | | | | |
| 86 mM | 1.5 | 4 | C | 39 | center | ⅛ | tap | No |
| NaCl pH | | | | | | | water | MBs |
| 7.2 | | | | | | | | |
| 87 mM | 1.5 | 5 | C | 39 | center | ⅛ | tap | No |
| NaCl pH | | | | | | | water | MBs |
| 7.2 | | | | | | | | |
| 85 mM | 1.5 | 2 | C | 39 | center | ⅛ | ice | No |
| NaCl pH | | | | | | | water | MBs |
| 7.2 | | | | | | | | |
| 85 mM | 1.5 | 3 | C | 39 | center | ⅛ | ice | No |
| NaCl pH | | | | | | | water | MBs |
| 7.2 | | | | | | | | |
| 85 mM | 1.5 | 4 | C | 39 | center | ⅛ | ice | No |
| NaCl pH | | | | | | | water | MBs |
| 7.2 | | | | | | | | |
| 85 mM | 1.5 | 5 | C | 39 | center | ⅛ | ice | No |
| NaCl pH | | | | | | | water | MBs |
| 7.2 | | | | | | | | |
| 85 mM | 1.5 | 6 | C | 39 | center | ⅛ | ice | No |
| NaCl pH | | | | | | | water | MBs |
| 7.2 | | | | | | | | |
| 85 mM | 1.5 | 7 | C | 39 | center | ⅛ | ice | No |
| NaCl pH | | | | | | | water | MBs |
| 7.2 | | | | | | | | |
| Distilled | 10 | 30 sec, | NC | 59, 80 | center, | ½ | NB | No |
| water | | 25 sec | | | surface | | | MBs |
| PBS pH | 10 | 30 sec, | NC | 59, 80 | center, | ½ | NB | No |
| 7.4 | | 25 sec | | | surface | | | MBs |
| 80 mM | 10 | 30 sec, | NC | 59, 80 | center, | ½ | NB | No |
| NaCl | | 25 sec | | | surface | | | MBs |
| 145 mM | 10 | 30 sec, | NC | 59, 80 | center, | ½ | NB | No |
| NaCl | | 25 sec | | | surface | | | MBs |
| Distilled | 10 | 1, 1, 1 | NC | 60, 80, 80 | center, | ½ | NB | No |
| water | | | | | surface, | | | MBs |
| | | | | | surface | | | |
| PBS pH | 10 | 1 | NC | 80 | surface | ½ | NB | No |
| 7.4 | | | | | | | | MBs |
| 80 mM | 10 | 1, 1 | NC | 60, 80 | bottom, | ½ | NB | gelled |
| NaCl | | | | | surface | | | |
| 145 mM | 10 | 30 sec, | NC | 60, 80 | bottom, | ½ | NB | No |
| NaCl | | 30 sec | | | surface | | | MBs |

Since lyophilized HSA did not yield robust microbubbles, a commercially available 5% HSA solution was tried (Plasbumin®-5, Bayer Corp., Indiana). This pre-prepared solution contains stabilizing agents (0.004 M sodium coprolite, 0.004 M acetylthryptophan). The initial conditions explored were the following, as shown in Table 3.

TABLE 3

| | | | Son. | | Son. | | Probe | | |
|---|---|---|---|---|---|---|---|---|---|
| | | Volume | time | Cont/Not | intensity | Horn tip | size | | |
| Sample | Solvent | (mL) | (min) | cont | (%) | position | (in.) | Bath | Result |
| 1 | Plasbumin ®-5 | 10 | 30 sec, | NC | 60, 60 | center, | ½ | NB | No |
| | | | 30 sec | | | surface | | | MBs |
| 2 | Plasbumin ®-5 | 10 | 30 sec, | NC | 60, 80 | center, | ½ | NB | MBs |
| | | | 30 sec | | | surface | | | low |
| | | | | | | | | | yield |
| 3 | Plasbumin ®-5 | 10 | 1 | C | 80 | surface | ½ | NB | MBs |
| | | | | | | | | | low |
| | | | | | | | | | yield |
| 4 | Plasbumin ®-5 | 10 | 1 | C | 100 | surface | ½ | NB | MBs |
| 5 | Plasbumin ®-5 | 10 | 1.5 | C | 80 | surface | ½ | NB | MBs |

Stable microbubbles were prepared. Even though all conditions yielded microbubbles, the microbubbles formed in sample 1 dissolved into the HSA solution after 24 h. Samples 2 and 3 still had microbubbles after 24 h but in a much lower yield than samples 4 and 5.

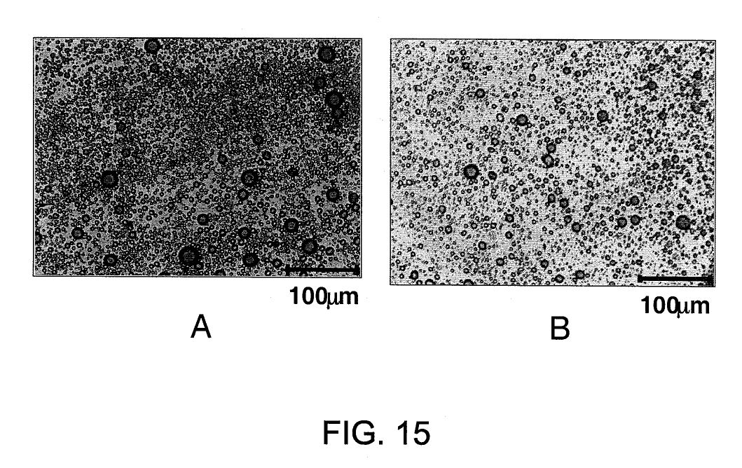

The microbubbles prepared were visually comparable to the benchmark selected Optison®. The size of the microbubbles was characterized by light microscopy (Olympus confocal microscope, BX51 Model, transmission mode) and the microbubble size distribution was characterized using a Particle Sizer (Beckman-Coulter, laser diffraction analyzer LS 100, CA). FIG. 15 shows that the population of microbubbles prepared is very comparable to the standard Optison®, where the mean size is in the range of 10 μm. These results were also reproduced by preparing microbubbles in a smaller scale, using 1 mL of Plasbumin®-5 instead of 10 mL. For such volumes, a stepped microtip and a tapered microtip were used.

The conditions tested are listed in Table 4. Conditions 1-6 were tried using the stepped microtip. For each sample, the microbubble yield was low. Conditions 1-5 and 7 were tried using the tapered microtip. For samples 1-2 the results were comparable to the results obtained when using the stepped microtip. However, conditions of samples 3-5 and 7 yielded a thick layer of microbubbles.

TABLE 4

| | | | | Son. | | Probe | | |
|---|---|---|---|---|---|---|---|---|
| | | Vol. | Son. | intensity | Horn tip | size | | |
| Sample | Solvent | (mL) | time | (%) | position | (in.) | Bath | Result^{a} |
| 1 | Plasbumin ®-5 | 1 | 1 min | 40 | surface | ⅛ | NB | MBs |
| | | | | | | | | low |
| | | | | | | | | yield |
| 2 | Plasbumin ®-5 | 1 | 30 sec, | 40 | center, | ⅛ | NB | MBs |
| | | | 30 sec | | surface | | | low |
| | | | | | | | | yield |
| 3 | Plasbumin ®-5 | 1 | 1.5 min | 40 | surface | ⅛ | NB | MBs |
| 4 | Plasbumin ®-5 | 1 | 45 sec, | 40 | center, | ⅛ | NB | MBs |
| | | | 45 sec | | surface | | | |
| 5 | Plasbumin ®-5 | 1 | 2 min | 40 | surface | ⅛ | NB | MBs |
| 6 | Plasbumin ®-6 | 1 | 75 sec, | 40 | center, | ⅛ | NB | — |
| | | | 1 min, | | rest, | | | |
| | | | 75 sec | | surface | | | |
| 7 | Plasbumin ®-7 | 1 | 76 sec, | 40 | center, | ⅛ | NB | MBs |
| | | | 2 min, | | rest, | | | |
| | | | 75 sec | | surface | | | |

### Example 2

### Labeling of Microbubbles Scaffolds

Covalent labeling of the microbubble scaffold could be done either before or after the formation of the microbubble. Considering the stability of the microbubbles, labeling prior to the formation of the microbubbles seemed more attractive. This approach allows the use of purified labeled scaffolds before making the microbubbles, avoiding the presence of excess fluorescent free dye adsorbed on the bubbles. Hence, this approach eliminates the need for extensive microbubble wash to remove excess dye, which may result in very low microbubble yield. In addition, pre-labeling the scaffolds that may eventually form the microbubble would allow for more control in terms of degree of labeling, which is a factor to consider when exploring the space that may provide the right separation of dyes to achieve the FRET phenomenon that could potentially allow for fluorescence modification.

### Example 2A

### Labeling of ST68 Microbubbles Scaffolds

The ST68 system is composed of both Tween 80 and Span 60. Tween 80 was selected as the component to be labeled with the fluorescent dye since it contains primary hydroxyl groups that may be easily modified. In addition, Tween 80 is the hydrophilic component of the ST68 system. Modification of the hydrophilic component with a water-soluble dye may minimize the distortion of the hydrophilic/hydrophobic balance needed for the formation of the microbubbles. The dye selected for labeling was the monoreactive NHS ester of Cy5.5 (GE Healthcare). Cy5.5 has a max absorbance at λₘₐₓ=675 nm, a max fluoresce emission at λₘₐₓ=694 nm. Some of the advantages of Cy dyes are their fluorescence in the near IR region, high extinction coefficient, water solubility, good quantum yields and photostability. Instead of selecting a set of donor and acceptor, the system was simplified by selecting Cy5.5, since it self-quenches at high concentrations.

Two different linkers that differ in length were initially selected for the modification of Tween 80. However, acidic conditions for deprotection of amine group of linkers after their conjugation onto Tween 80 caused hydrolysis of Tween 80 at ester bond that connects its polar head to apolar tail.

To avoid hydrolysis a new linker was selected. The Cbz-β-Ala-OH linker may be deprotected under hydrogenolysis conditions. These conditions may reduce the double bond of the alkyl chain. However, this change may not alter the assembly properties.

The first coupling step was done by dissolving equal molar ratios of Tween 80 and the linker with CH₂Cl₂, followed by the addition of 1.5 equivalents of DCC. The reaction mixture was stirred for 5 h and the DCU byproduct was filtered off using a glass filter. The product was purified through column chromatography (CH₂Cl₂/MeOH, 9:1). ¹H-NMR confirmed presence of the linker. Then, the product (0.5 g) was dissolved in MeOH and 0.1 g of 10% Pd/C was added to solution. The hydrogen donor 1,4-hexacyclodiene (6 mL) added and stirred at 60° C. under N₂ atmosphere for 5.5 hours. The Pd/C was removed by filtration using glass filter with a Celite pad. The solvent of the filtrate was evaporated under high vacuum and light yellow residue was obtained. ¹H-NMR showed full cleavage of the Cbz group and only one spot is observed by TLC. The product of reaction was dissolved with 1M NaHCO₃, followed by the addition of a DMSO solution of NHS-Cy5.5. The mixture was stirred in the dark at room temperature for 24 h. The reaction product was purified using a size exclusion PD-10 column (GE Healthcare). The high molecular weight band was collected, frozen, and lyophilized.

### Example 2B

### Labeling of HSA Microbubbles Scaffolds

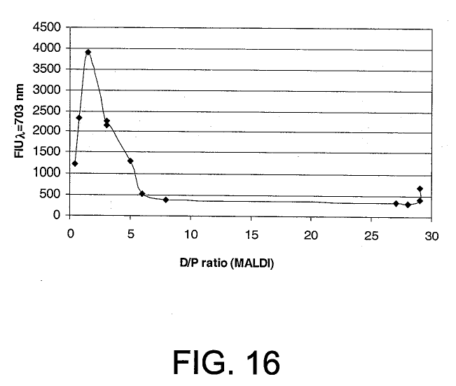

For the labeling of HSA, a library of HSA-Cy5.5 conjugates was prepared by changing the dye/protein ratio used in the reaction mixture. An example of the experimental procedure is as follows: 20 mg of lyophilized HSA was dissolved with 0.8 mL of freshly prepared 0.1 M NaHCO₃ (pH 8.4) solution. A solution of NHS-Cy5.5 was prepared with anhydrous DMSO at a concentration of 10 mg/mL. An aliquot of the NHS-Cy5.5 solution was added to the protein solution and stirred for 4 h. The reaction mixture was transferred to an ultrafiltration tube Amicon Ultra4 (GE Healthcare) with MWCO of 30 KDa and used as suggested by vendor. The samples were washed 4 times. This procedure removed most excess of the free dye. In a final purification step, the concentrate from the Amicon filter was eluted through a size exclusion PD-10 column to remove remainder small MW dye. The high molecular weight fraction was collected, frozen, and lyophilized. A library with different dye/protein ratio was prepared (FIG. 16). The fluorescence of the different conjugates at equal concentrations was monitored at λₘₐₓ=703 nm. FIG. 16 shows the different conjugates prepared with different D/P ratios. The fluorescence increases as a function of dye content, but then decreases once a limit is reached due to self-quenching.

The D/P quantification of this system could not determined by traditional methods based on UV absorption, as commonly used for this purposes. The results obtained would vary considerably as varying the concentration of the solutions and numbers with no physical meaning (negative numbers) would be obtained. Therefore, MALDI-MS was used to determine the conjugate mass and D/P was determined by mass difference.

### Example 3

### Production of Fluorescent Microbubbles

The surfactant formulation used for the preparation of ST68 fluorescently labeled microbubbles was prepared. Aliquots of 10 mL of the surfactant formulation were mixed with 1 mg of labeled scaffold. As a control, 10 mL of the formulation were mixed with free Cy5.5 dye. A 0.5-inch probe was used, and the samples were sonicated at 100% intensity for 2.5 min while keeping solution immersed in ice bath. For the preparation of HSA fluorescent microbubbles, 10 mL aliquots of Plasbumin®-5 were mixed with 1 mg of Cy5.5-HSA conjugate. The mixture was sonicated with a 0.5-inch probe using 80% intensity for 1.5 minutes. The solution was not immersed in a water bath. A mixture of HSA and free Cy5.5 dye was also prepared as a control.

The fluorescence images of the microbubbles were obtained using an Olympus fluoview FV300 laser scanning confocal microscope, modified to accept light from a 3.0 mW 680-nm laser diode (Edmund Scientific). A 10-× objective (UPLAPO10×, N.A. 0.40) was employed that produced an image field for the XY galvanometer mirror scanners roughly 280-sqaure microns in size. Cy5.5-based fluorescence intensity was detected by a Hamamatsu photomultiplier tube positioned behind a confocal pinhole and 700-nm long-pass filter.

After preparation of the microbubbles, the first visual observation was that only the Cy5.5-HSA mixture showed a microbubble layer that was colored. On the other hand the ST68 bubbles and the controls showed a blue solution and colorless bubble layer.

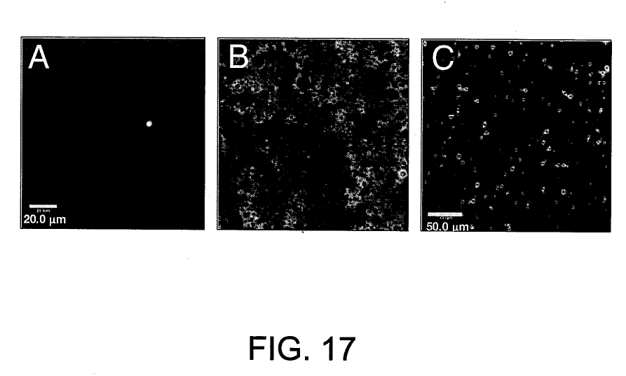

For the ST68 microbubbles, the fluorescence did not seem to be incorporated on the shell of the microbubble, but instead it remained in solution. A possible explanation for this result is that the solubility properties of the labeled Tween 80 scaffold changed enough to disturb the fine balance of hydrophobic/hydrophilic properties necessary for successful incorporation into the shell. On the other hand, fluorescent microbubbles using HSA as a scaffold were successfully prepared. Also notice that the control shows the same pattern as for the ST68 microbubbles, the bubbles are not fluorescent, but the solution contains free fluorescent dye, as expected. FIG. 17 shows confocal microscope pictures of microbubbles in fluorescence mode: (Panel A) Cy5.5-ST68 microbubbles, (Panel B) Control: HSA+free Cy5.5, and (Panel C) Cy5.5-HSA fluorescent microbubbles.

### Example 4

### Evaluation of Microbubbles

Once fluorescent microbubbles were successfully prepared, the next step was the evaluation of their ability to modulate fluorescence upon changes in size. The goal was to use pressure to induce a size change in the bubbles causing a change the total emitted fluorescence. A model consisting of a microchannel pressure chamber to measure individual bubbles using scanning laser confocal microscopy was setup.

A pressure chamber was constructed of two pieces of polycarbonate. Each piece was about the size of a microscope slide, 1 inch by 3 inches. The pieces were connected with a piece of double-sided tape with a long, thin strip removed from the middle of the tape. The window was approximately 1 mm wide and 30 mm long. The top piece of polycarbonate had 2 small holes drilled thorough it where the ends of the window in the tape were located to create a microchannel the size of the window in the tape. This top piece had nanoports (Upchurch Scientific, N-333) attached above each hole to connect 1/16 inch OD tubing to the microchannel. The perimeter gap of the two polycarbonate pieces was sealed with epoxy. Both ends of the tubing were connected to a pneumatic pressure controller (Druck Limited, DPI 530). The pressure controller used an air pressure source at 90 p.s.i. and an external vacuum source (Cole-Parmer) to maintain the pressure within the tube and microchannel to the pressure level selected by the user. The tubing was connected to a T-connector with equal length of tubing connected to each side of the microchannel to minimize the movement of the bubbles with changing pressures during imaging. The microchannel was mounted on an Olympus Fluoview laser scanning confocal microscope, employing an external 670-nm laser.

The fluorescent bubbles were imaged in a closed system at different pressures. Air bubbles in the microchannel were able to confirm the changing pressure within the channel; however, the radius of the fluorescent microbubbles did not change a measurable amount.

In MATLAB, a Hough transformation was used to identify fluorescent microbubbles in the image and measure their radius; however, several challenges were identified. The laser confocal microscope has a very small depth of focus so the images were cross-sections of the bubbles instead of the true radius. In addition, not all bubbles could be measured in each image taken at each pressure due to movement of the bubbles caused by pressure changes.

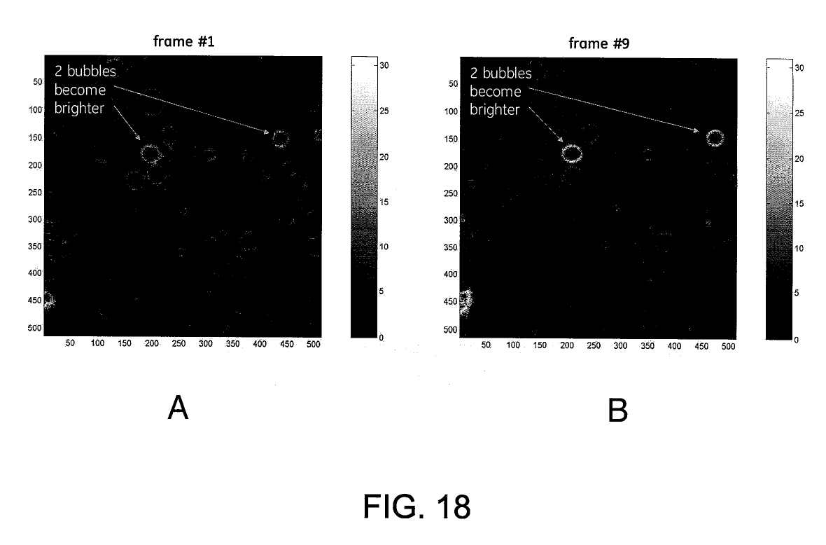

The laser scanning confocal microscope uses a raster scan to generate an image. This requires long exposure times with high fluence from the laser. The typical experiment took roughly 10 scans in approximately 5 minutes. Photobleaching of the bubbles was observed. The laser excitation employed to produce images led to photobleaching in all of the formulations tested. FIG. 18 depicts an example of this phenomenon.

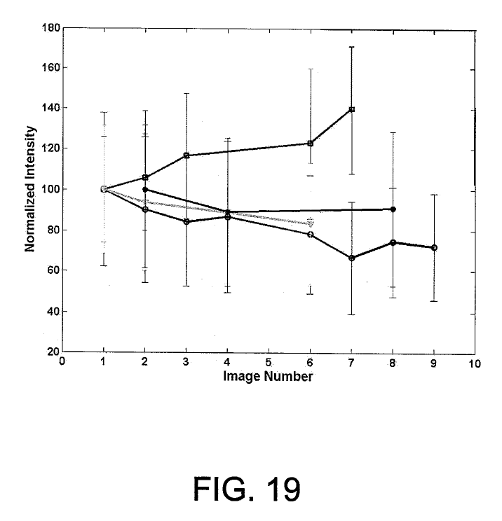

A series of images was taken to watch the effects of photobleaching at atmospheric pressure. An interesting photobleaching effect was observed as fluorescence intensity increased or decreased for different microbubbles within the same formulation. The two bubbles that are identified in frame #1 (FIG. 18) have greater fluorescence intensity in frame #9 whereas the other microbubbles in the image have less fluorescence intensity. The normalized average fluorescence intensities of four bubbles in the images of FIG. 18 are plotted in FIG. 19. The graph shows three of four bubbles selected decreased in intensity while one increased.

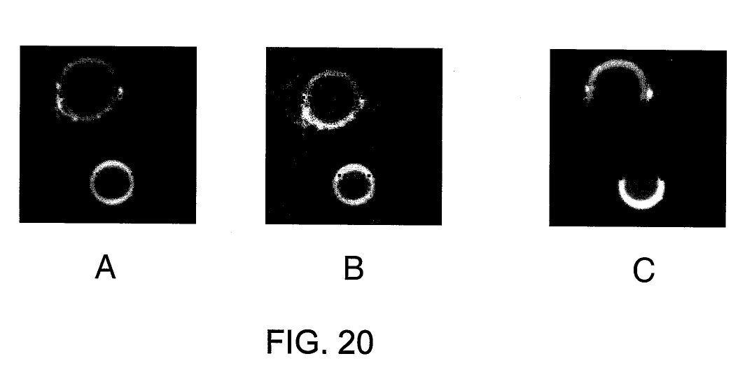

A closer look was taken at this phenomenon by photobleaching a portion of an individual microbubble (FIG. 20). A higher magnification objective lens was used and only a portion of the image was scanned, two halves of two separate bubbles. The results show increased fluorescence upon photobleaching, followed by decreased fluorescence with additional photobleaching. A possible explanation of the effects is that fluorescence increases upon photobleaching in the case of microbubbles that were initially self-quenched due to their high local concentration of dyes. However, in the case of non self-quenched or partially photobleached microbubbles, the intensity decreases due to decrease in number of active fluorescent dyes.

The observation of this phenomenon was key. It supports the idea of fluorescent modulation upon changes in local concentration of dyes on the microbubble surface.

While only certain features of the invention have been illustrated and described herein, many modifications and changes may occur to those skilled in the art. It is, therefore, to be understood that the appended claims are intended to cover all such modifications and changes as fall within the true spirit of the invention.

1. A deformable particle, comprising:
(i) a shell encasing an internal substance that expands or contracts in response to an ultrasonic stimulus; and
(ii) at least one FRET pair comprising a fluorescent component and a quenching component, wherein the fluorescent component and a quenching component are positioned relative to each other so that the FRET pair emits an enhanced optical signal when the deformable particle transitions from a neutral conformation to a deformed conformation.

2. The deformable particle of claim 1, wherein the FRET pair the optical signal is enhanced when the deformable particle is in an expanded conformation and the FRET pair members are positioned at a distance greater than the characteristic distance.

3. The deformable particle of claim 2, wherein the optical signal is enhanced at least two fold.

4. The deformable particle of claim 1, wherein the internal substance comprises a gas, a fluid, or a combination of gas and fluid that expands in response to an ultrasound transmission.

5. The deformable particle of claim 1, wherein the internal substance comprises air, sulfur hexafluoride, or perfluorocarbon.

6. The deformable particle of claim 1, wherein the perfluorocarbon comprises perfluoropropane, perfluorobutane, perfluoropentane, or perfluorohexane, or perfluorocarbon gaseous precursor.

7. The deformable particle of claim 1, wherein the shell comprises an amphiphilic substance.

8. The deformable particle of claim 1, wherein the amphiphilic substance comprises a polymer, a protein, or a surfactant.

9. The deformable particle of claim 8, wherein the protein comprises mammalian serum albumin.

10. The deformable particle of claim 8, wherein the protein comprises human serum albumin.

11. The deformable particle of claim 8, wherein the surfactant comprises a detergent selected from C12-sorbitan-E20; Polysorbate 20; Polysorbate 80; C16-sorbitan-E20; or C18-sorbitan-E20.

12. The deformable particle of claim 1, wherein fluorescent component comprises a fluorophore selected from indocyanine green, cyanine 5.5, fluorescein, rhodamine, yellow fluorescent protein, green fluorescent protein, and derivatives thereof.

13. The deformable particle of claim 1, wherein both members of the FRET pair are positioned on the outer surface of the shell.

14. The deformable particle of claim 1, wherein both members of the FRET pair are positioned within the shell.

15. The deformable particle of claim 1, wherein one member of the FRET pair is positioned on the outer surface of the shell and the other member of the FRET pair is positioned within the shell.

16. The deformable particle of claim 1, wherein the concentration of the quenching component and the concentration of the fluorescent component are substantially equivalent.

17. The deformable particle of claim 1, wherein the shell further comprises a binder capable of binding to a predetermined target

18. The deformable particle of claim 17, wherein the binder comprises at least one of antibodies, ligands, or nucleic acids.

19. A combined modality imaging system, comprising:
a deformable particle;
an ultrasound imaging device; and
an optical imaging device; wherein the ultrasound imaging device comprises an ultrasound probe, a data acquisition and processing system, and an operator interface.

20. The combined modality imaging system of claim 19, wherein the ultrasound imaging device comprises an ultrasound probe including at least one of an ultrasound transducer, a piezoelectric crystal, and a micro-electro mechanical system device.

21. The combined modality imaging system of claim 19, wherein the ultrasound probe comprises an electromagnetic excitation source and an electromagnetic radiation detector.

22. The combined modality imaging system of claim 19, wherein the ultrasound probe comprises a multitude of electromagnetic radiation detectors.

23. The combined modality imaging system of claim 19, wherein the ultrasound imaging device comprises a display module to provide a visual display of an ultrasound image in at least one of gray-scale mode and color mode.

24. The combined modality imaging system of claim 19, wherein the ultrasound imaging device comprises a printer module to provide a hard copy of an ultrasound image in at least one of gray-scale mode and color mode.

25. The combined modality imaging system of claim 19, wherein the optical imaging device comprises an electromagnetic excitation source adapted to emit electromagnetic radiation into the subject and an electromagnetic radiation detector adapted to detect electromagnetic radiation emitted from the contrast agent disposed within the subject.

26. The combined modality imaging system of claim 19, wherein the optical imaging device comprises a data acquisition module, a data processing module, and an operator interface.

27. The combined modality imaging system of claim 19, wherein the electromagnetic excitation source comprises at least one radiation transmitting device selected from a group consisting of a solid-state light emitting diode, an organic light emitting diode, an arc lamp, a halogen lamp, and an incandescent lamp.

28. The combined modality imaging system of claim 19, wherein the electromagnetic excitation source comprises at least one radiation transmitting device adapted to emit electromagnetic radiation at least between the ranges of about 300 nanometers and about 2 micrometers.

29. The combined modality imaging system of claim 19, wherein the electromagnetic radiation detector comprises at least one detector selected from a group comprising a photo-multiplier tube, a charged-coupled device, an image intensifier, a photodiode, and an avalanche photodiode.

30. The combined modality imaging system of claim 19, wherein the optical imaging device comprises at least one fiber-optic channel adapted to convey the electromagnetic radiation from the electromagnetic excitation source to the focus area of the subject.

31. The combined modality imaging system of claim 19, wherein the optical imaging device comprises at least one fiber-optic channel adapted to convey the electromagnetic radiation emitted by the contrast agent to the electromagnetic radiation detector.

32. A method of use of a combined modality imaging system, the method comprising:
(a) administering the deformable particle of claim 1 to a subject;
(b) applying ultrasound waves into the subject toward a region of interest;
(c) applying electromagnetic radiation toward the region of interest;
(d) detecting ultrasound signals reflected from the region of interest;
(e) detecting electromagnetic radiation from deformable particle; and
(f) processing the detected ultrasound signals and the detected electromagnetic radiation.

33. The method of claim 32, wherein the processing step includes producing at least one co-registered image.

34. The method of claim 32, wherein applying ultrasound waves and detecting ultrasound signals comprises engaging an ultrasound probe with the subject, the ultrasound probe comprising at least one of an ultrasound transducer, a piezoelectric crystal, and a micro electro mechanical system device.

35. The method of claim 32, further comprising emitting electromagnetic radiation from the fluorescent component in response to emissions from an electromagnetic radiation based imaging device;
(a) modulating the geometry of the deformable particle in response to a pressure wave by an ultrasound imaging device; and
(b) decreasingly absorbing, with the quenching component, a portion of the electromagnetic radiation emitted by the fluorescent component in response to increasing the geometry of the deformable particle.
